(19) United States
(12) Patent Application Publication
(10) Pub. No.: US 2013/0060277 A1
(43) Pub. Date: Mar. 7, 2013

(54) Bilateral Wound Compress

(71) Applicant: Regina D'Andrea, Providence, RI (US)

(72) Inventor: Regina D'Andrea, Providence, RI (US)

(21) Appl. No.: 13/373,800

(22) Filed: Sep. 2, 2011

(51) Int. Cl. A61B 17/00

(52) U.S. Cl. 606/201

(57) ABSTRACT

I, Regina D'Andrea, hereby submit this application to patent the Bilateral Wound Compress. A bilateral wound compress is designed to apply constant pressure to a wound. The Bilateral Wound Compress consists of two blocks screwed together with the wounded body part in between.

### (2) CROSS-REFERENCE

| Patent # | Date Filed | Name of Inventors |
|---|---|---|
| 2,383,670 | Jul. 1, 1942 | Chester C. Moss, Jackson, Mich., assignor |
| | | to Walker Manufacturing Company of |
| | | Wisconsin, Racine, Wis., a corporation of |
| | | Wisconsin. |
| 1,597,525 | Jan. 11, 1924 | Arthur H. Knake, of St. James, Minnesota. |

### (3) STATEMENT REGARDING FEDERALLY SPONSORED RESEARCH OR DEVELOPMENT

There has been no federally sponsored research of development involved in the development of this design patent.

### (4) DESCRIPTION OF THE FIGURE(S) OF THE DRAWING

FIG. 1 is top view of block #1 and block #2 because they are identical.

FIG. 2 is a side view of block #1 and block #2 because they are identical.

FIG. 3 is a frontal view of the bilateral wound compress in operation.

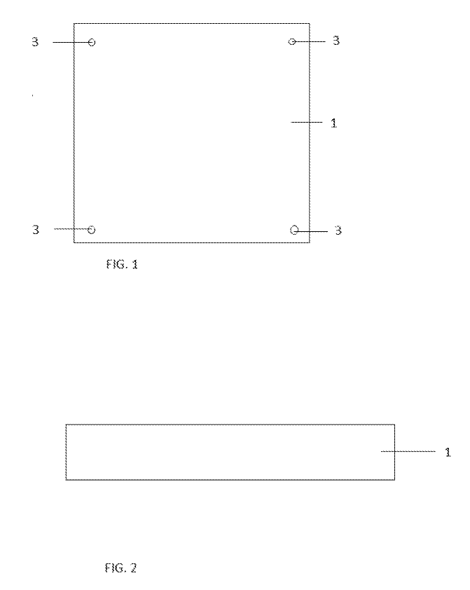

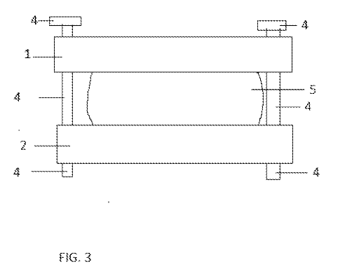

### DETAILED DESCRIPTION OF THE PREFERRED EMBODIMENT

Referring to FIG. 1 block #1 1 and block #2 1 are identical. Referring to FIG. 1, block #1 1 and block #2 1 are preferably formed of a hard material such as plastic or metal. Block #1 1 and block #2 may come in a different shape, dimensions, or thickness to fit the body part being treated. Block #1 1 and block #2 must have screw holes 3 along the outer perimeter and in identical places.

In operation, block #1 1 is to be placed directly on top of the wound. Block #2 1 must be place on the opposite side of the body part affected by the wound 5 so that the blocks are aligned symmetrically and the screw holes line up. Screws 4 are then screwed through the screw holes 3 block #1 1 and block #2 1 so that block #1 1 and block #2 1 are held together securely with the body part affected by the wound 5 in between block #1 1 and block #2 1. The screws may be tightened to achieve the degree of pressure required, or loosened to relieve pressure and increase circulation to the body part affected by the wound 5.

While the invention is susceptible to various modifications and alternative forms, specific embodiments thereof have been shown in the drawings and are herein described in detail. It should be understood, however, that the invention is not to be limited to the particular form disclosed, but to the contrary, the invention is to cover all modifications, equivalents, and alternatives falling within the spirit and scope of the appended claims.

### (5) Feature Description

### FIELD OF INVENTION

The present invention relates to applying pressure to bleeding wounds to stop them from bleeding.

### BACKGROUND OF THE INVENTION

Bleeding wounds require the application of constant pressure to stop bleeding. Some bleeding may be stopped with stitches but sometimes stitches are not an option. For example, doctors usually don't use stitches to close a wound after 8 hours because there is a chance they may seal in infection. Another example is if a patient is performing at-home care. Another example is if the wound is too large to be stitched. Stitches may be avoided if pressure stops bleeding within fifteen to twenty minutes. Currently, pressure is applied to dressed wounds by wrapping a pressure bandage around the wound. The process of wrapping the bandage tightly can be irritating to the wound site because the pulling of the bandage pulls on the wound, therefore preventing healing and possibly increasing bleeding. This also prevents the bandage from being applied tightly enough, which reduces pressure and reduces healing. This wrapping of the pressure bandage applies pressure from all sides rather than directly on the wound. The wound would benefit from a mechanism that would maximize pressure directly on the wound without pulling on the wound.

### SUMMARY OF THE INVENTION

One block of the Bilateral Wound Compress is placed directly on top of the wound. The second block is placed on the opposite side of the body part affected by the wound so that the blocks are aligned symmetrically. Screws are screwed through screw holes in the blocks so the blocks are held together in place with the body part in between the blocks. The screwed may be tightened to increase pressure on the wound or loosened to alleviate pressure and allow more circulation.

Other aspects and features of the present invention will become apparent from consideration of the following description taken in conjunction with the accompanying drawings.

What is claimed:

1. A bilateral wound compress comprises block #1 and block #2 with screw holes and screws to hold the two blocks together.

2. The device of claim 1, wherein there is padding or absorbant material such as batting material, foam padding, cotton, gauze, and the like, in between or around the two blocks.

3. The device of claim 1, wherein the two blocks are formed as a single unit.

4. The device of claim 1, wherein washers are places between the screws and the blocks.

5. The device of claim 1, wherein another binding mechanism such as a clamp, elastic band, nail, and the like, is used with or without the screws.
